(19) United States
(12) Patent Application Publication
(10) Pub. No.: US 2017/0235116 A1
(43) Pub. Date: Aug. 17, 2017

(54) LIGHTING ARRANGEMENT

(71) Applicant: Carsten JUNG, Asslar (DE)

(72) Inventor: Carsten JUNG, Asslar (DE)

(21) Appl. No.: 15/344,787

(22) Filed: Nov. 7, 2016

(30) Foreign Application Priority Data
Nov. 12, 2015 (DE) 10 2015 119 590.9

(51) Int. Cl. G02B 21/08; A61B 5/00; A61B 90/20; A61B 90/30; G02B 21/02; G02B 21/00

(52) U.S. Cl. CPC G02B 21/082; G02B 21/02; G02B 21/0012; A61B 90/20; A61B 90/30; A61B 5/0059

(57) ABSTRACT

Lighting arrangement for a diagnostic or surgical microscope has a light source, and the lighting arrangement defining an illuminating beam path. An illumination optics is arranged between the light source and an objective of the microscope which defines an imaging beam path, and in the beam path of the light source at least one deflection element is arranged for coupling in of the illuminating beam path into the imaging beam path. The illumination optics has at least one pancratic system. The lighting arrangement has, in the manner of a Köhler illumination, a collector lens arrangement which is arranged downstream of the light source in the beam direction of the light source, a field diaphragm arranged downstream of the collector lens arrangement, and an auxiliary lens arrangement arranged downstream of the field diaphragm, and the auxiliary lens arrangement has at least one pancratic system.

### CROSS-REFERENCE TO RELATED APPLICATIONS

This application claims the priority of German Application No. 10 2015 119 590.9, filed Nov. 12, 2015, which is incorporated herein by reference.

### FIELD OF THE INVENTION

This invention relates to a lighting arrangement for a diagnostic or surgical microscope. In particular, in the case of diagnostic or surgical microscopes there is a requirement to sufficiently illuminate an object to be examined by use of the microscope. For this purpose a lighting arrangement is used, of which the illuminating beam path is blended, for example, by means of a deflecting element into the observation beam path defined by the objective of the microscope. At the same time there is a requirement to achieve a homogeneous illumination.

### BACKGROUND OF THE INVENTION

Lighting arrangements for microscopes are known, for example, from DE 10 2007 027 084 A1, DE 10 2011 086 178 A1, DE 10 2006 017799 A1 and DE 101 44 062 A1.

DE 103 47 732 A1 discloses a lighting arrangement for a microscope which has a light source and an illumination optics which is or can be arranged between the light source and an objective of the microscope.

JP 2011/242609 A discloses a microscope with a lighting arrangement, wherein an a focal pancratic system is arranged in the imaging beam path of the microscope.

A similar microscope is also disclosed by US 2007/0091939 A1.

EP 1 153 569 A2 discloses a surgical microscope which has an illuminating beam path, wherein dimming of the illumination on the object is achieved by removing or changing the position of an optical component.

DE 103 55 523 A1 discloses a stereo microscope, wherein both in the imaging beam path and also in the illuminating beam path a zoom system is arranged in order to produce illumination which is adapted to the zoom factor of the illuminating team path.

US 2009/0091822 A1 discloses a lighting arrangement of the type in question which has a light source, wherein the lighting arrangement defines an illuminating beam path. An illumination optics is arranged between the light source and an objective of the microscope which defines an imaging beam path, wherein in the beam path of the light source at least one deflection means is provided for the coupling in of the illuminating beam path into the imaging beam path and wherein the illumination optics has at least one pancratic system.

A similar lighting arrangement is also disclosed by US 2002/0159143 A1.

### OBJECTS AND SUMMARY OF THE INVENTION

An object of the invention is to provide a lighting arrangement which enables improved illumination.

This object is achieved by the invention set forth herein.

The invention includes a lighting arrangement for a diagnostic or surgical microscope, the lighting arrangement having a light source, wherein the lighting arrangement defines an illuminating beam path. It further includes an illumination optics, the illumination optics is or can be arranged between the light source, and an objective of the microscope, which defines an imaging beam path. In the beam path of the light source at least one deflection means is provided for coupling in of the illuminating beam path into the imaging beam path. The illumination optics has at least one pancratic system. Also, the lighting arrangement has, in the manner of a Köhler illumination, a collector lens arrangement which is arranged downstream of the light source in the beam direction of the light source, a field diaphragm arranged downstream of the collector lens arrangement, and an auxiliary lens arrangement arranged downstream of the field diaphragm; and the auxiliary lens arrangement has at least one pancratic system.

The invention likewise includes a lighting arrangement for a diagnostic or surgical microscope, the lighting arrangement having a light source, wherein the lighting arrangement defines an illuminating beam path. It further includes an illumination optics, the illumination optics is or can be arranged between the light source, and an objective of the microscope, which defines an imaging beam path. In the beam path of the light source at least one prism or mirror is provided for coupling in of the illuminating beam path into the imaging beam path. The illumination optics has at least one pancratic system. Also, the lighting arrangement has, in the manner of a Köhler illumination, a collector lens arrangement which is arranged downstream of the light source in the beam direction of the light source, a field diaphragm arranged downstream of the collector lens arrangement, and an auxiliary lens arrangement arranged downstream of the field diaphragm; and the auxiliary lens arrangement has at least one pancratic system.

The invention is based on the idea of configuring the lighting arrangement so that, if required, an increase in the illumination intensity is enabled in a simple manner while simultaneously maintaining the homogeneity of the illumination along the illuminated surface in the observation field.

Starting from this basic idea, the invention uses Köhler illumination, wherein the light source is imaged by means of a collector lens arrangement into the plane of a field diaphragm (aperture diaphragm), and an auxiliary lens arrangement is arranged downstream of the field aperture.

The auxiliary lens arrangement of the Köhler illumination has a pancratic system (zoom lens) according to the invention. The pancratic system enables a change in the focal length of the illumination optics. If at the shortest focal length of the pancratic system the illumination optics homogeneously illuminates a specific area in the observation field, the illuminated area decreases with the variation of the focal length, wherein the illumination intensity increases simultaneously. If, for example, during adjustment of the focal length, the focal length of the pancratic system is lengthened by a factor of 2, the illuminated field diameter is decreased linearly by the same factor, wherein the geometric illumination intensity increases by a factor of 4.

The invention makes it possible, if required, to substantially increase the illumination intensity of the lighting arrangement in the observation field.

The invention also makes it possible in particular to employ light-emitting diodes with a relatively low illumination intensity as a light source of a lighting arrangement in a diagnostic or surgical microscope. Thus the invention makes full use of the advantages of light-emitting diodes for use in lighting arrangements for microscopes.

The zoom facto the pancratic system is selectable within wide limits according to the particular requirements.

Similarly, the design of the optics of the pancratic system is selectable within wide limits according to the particular requirements.

In the lighting arrangement according to the invention, any suitable light sources can be used, for example halogen light sources or fiber-optic light sources. An extraordinarily advantageous further embodiment of the invention in this respect provides that the light source has at least one light-emitting diode. In this way the advantages of light-emitting diodes are made useful for lighting arrangements for microscopes.

Another extraordinarily advantageous further embodiment of the invention provides that the lighting arrangement has at least one optical device for reducing reflections of the illumination light on the objective of the microscope. In this way, for the purpose of an accurate observation, negative effects of the illumination light on the observation beam path of the microscope, which originate from reflections of the illumination light, are avoided or at least decreased.

In the aforementioned embodiment the optical device for reducing reflections can be configured according to the respective requirements in a variety of ways. In order to configure this device particularly simply and thus cost-effectively, an advantageous further embodiment provides that the optical device for reducing reflections has at least one pinhole diaphragm.

In the aforementioned embodiment the pinhole diaphragm has at least one opening for the illumination main beam path of the lighting arrangement.

Another advantageous further embodiment of the invention provides that the deflection means is a prism or a mirror.

Advantageously, the field diaphragm can be designed as an iris diaphragm, as provided by another further embodiment of the invention.

A microscope according to the invention, in particular a diagnostic or surgical microscope, is disclosed herein. The microscope includes the lighting arrangement according to the invention.

In the context of the invention an optics is understood to be an arrangement which has at least one optically effective component, in particular at least one optical lens.

The invention is explained further below on the basis of an embodiment with reference to the appended highly schematic drawings.

Relative terms such as left, right, up, and down are for convenience only and are not intended to be limiting.

### BRIEF DESCRIPTION OF THE DRAWINGS

In the drawings:

FIG. 1 shows, highly schematically, an exemplary embodiment of a microscope according to the invention provided with an embodiment of a lighting arrangement according to the invention;

FIG. 2 shows, highly schematically, the lighting arrangement used in the microscope according to FIG. 1 at a first focal length of the pancratic system; and

FIG. 3 shows, in the same way as FIG. 2, the lighting arrangement with a second focal length of the pancratic system.

### DETAILED DESCRIPTION OF THE INVENTION

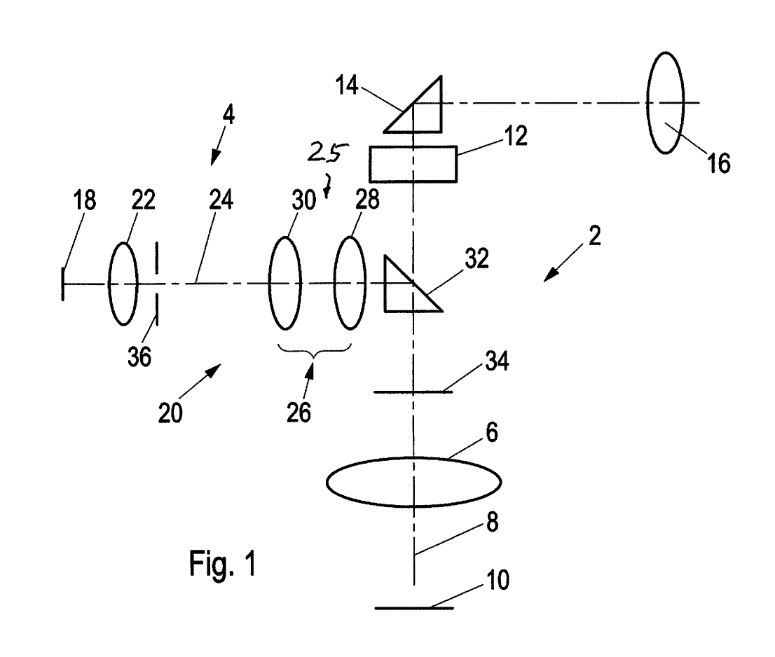

In FIG. 1, a highly schematic exemplary embodiment of a microscope according to the invention is provided with an exemplary embodiment of a lighting arrangement 4 according to the invention. Depending upon the construction of the microscope 2, the lighting arrangement 4 can be structurally integrated therein or can be formed as a separate assembly which can be connected to the microscope 2.

The microscope 2 may be in particular a diagnostic or surgical microscope.

The microscope 2 has an objective 6 (front objective), of which the optical axis defines an observation beam path 8 of the microscope 2 symbolized in FIG. 1 by a dash-dot line. From an object 10 to be observed, the observation beam path. 8 leads via a magnification changer 12 and a deflecting prism 14 to an eyepiece 16 of the microscope 2.

In the illustrated embodiment the lighting arrangement 4 according to the invention has a light source 18 in the form of a light-emitting diode.

The lighting arrangement 4 also has an illumination optics 20 with a collector lens arrangement 22 arranged downstream of the light source 18 in the beam direction, the optical axis of which defines a beam path 24 likewise symbolized in FIG. 1 by a dash-dot line. A field diaphragm 36 is arranged downstream of the collector lens arrangement 22 in the beam direction of the illuminating light, and an auxiliary lens arrangement 25 acting as a condenser is preferably arranged downstream of the field diaphragm 36 in the beam direction of the illumination light.

According to the invention, the auxiliary lens arrangement 25 of the illumination optics 20 has a pancratic system 26 (zoom lens) comprising a variator 28 and a compensator 30, wherein the pancratic system 26 is arranged downstream of the collector lens arrangement 22 in the beam direction of the light source 18.

For blending in of the illuminating beam path 24 into the observation beam path 8 of the microscope 2 deflection element or deflector 32 is provided which is arranged downstream of the pancratic system 26 and is formed by a prism in this exemplary embodiment. However, the deflection element or deflector 32 can also be formed by a mirror.

The basic structure and the basic mode of operation of a pancratic system are generally known to the person skilled in the art and therefore are not explained in greater detail here.

As can be seen from FIG. 1, the deflection means 32 deflects the illuminating beam path 24 by 45° so that the illuminating beam path 24 is blended into the observation beam path 8.

An optical device 34 for reducing reflections of the illumination light on the objective 6 is arranged in the beam direction between the deflection means 32 and the objective 6 of the microscope 2. In the illustrated exemplary embodiment, this device 34 is formed by an optical pinhole diaphragm. In addition to an opening with a circular boundary for the main beam path of the illuminating light, the optical pinhole diaphragm may, for example, have openings for the off-axis beam path of the illuminating light. The last-mentioned openings can have a circular basic shape, wherein it has proved advantageous if, in a deviation from the circular basic shape, they are slightly asymmetrical.

A field diaphragm 36, which may be formed in particular as an iris diaphragm, is arranged in the beam path of the light source 18 in the beam direction between the collector lens arrangement 22 and the auxiliary lens arrangement including the pancratic system 26. The collector lens arrangement 22 forms with the field diaphragm 36 and the auxiliary lens arrangement a Köhler illumination, in which the collector lens arrangement 22 images the light source into the plane of the aperture diaphragm 36. The structure of a Köhler illumination is generally known to the person skilled in the art and therefore is not explained in greater detail here.

In order to simplify the illustration, in the drawings the different optics (objective 6, eyepiece 16, collector lens arrangement 22) or parts thereof (variator 28, compensator 30) are symbolized in each case by an individual lens in the form of a convergent lens. It will be understood by the person skilled in the art that, instead of the individual convergent lens to be understood symbolically, a plurality of lenses as well as a combination and different lenses can form the respective optical assembly.

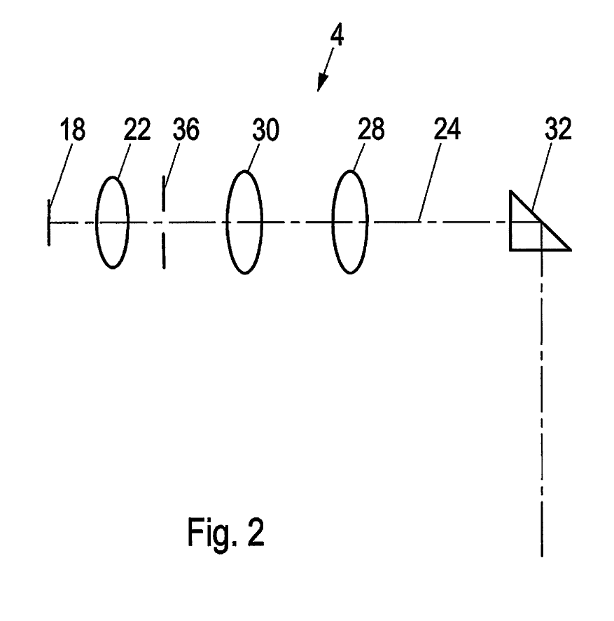

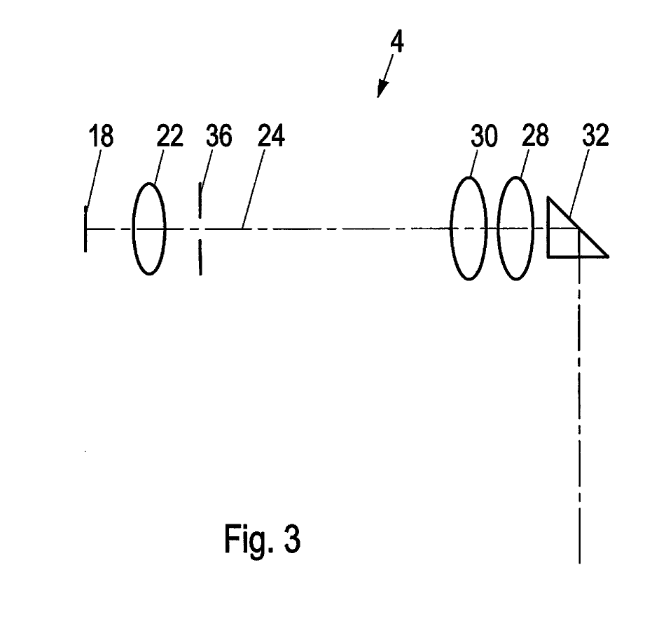

The mode of operation of the lighting arrangement according to the invention is explained in greater detail below with reference to FIG. 2 and 3, which in each case show the lighting arrangement 4 by itself.

FIG. 2 shows the pancratic system 26 of the illumination optics 20 in a focal length setting which corresponds to the shortest focal length of the pancratic system 26. In this focal length setting the observation field of the microscope 2 is homogeneously illuminated according to the illumination intensity of the light source 18.

In order to increase the illumination intensity in the central region of the observation field, the pancratic system 26 is adjusted for lengthening of the focal length. FIG. 3 shows the focal length setting of the pancratic system 26 which corresponds to the longest focal length. If the pancratic system 26 has, for example, a zoom factor of 2, the focal length in the focal length setting illustrated in FIG. 3 is twice as long as in the focal length setting illustrated in FIG. 2. Furthermore, since the illumination is homogeneous in the part of the observation field which is then illuminated, the geometric illumination intensity is increased by a factor of 4 by the adjustment of the focal length.

Thus the lighting arrangement according to the invention makes it possible, if required, to substantially increase the illumination intensity in the observation field. Thus light-emitting diodes can also be used as the light source, of which the illumination intensity is lower than the maximum illumination intensity required in the observation field would necessitate for the purpose of sufficient illumination. Thus the invention enables the use of relatively cost-effective light sources and simultaneous maintenance of a homogeneous illumination and guarantees sufficient illumination intensity in the observation field.

Suitable adjusting mechanisms for actuating the zoom function of the pancratic system 26 are generally known to the person skilled in the art and therefore are not explained in greater detail here.

In lighting arrangements without a pancratic system, which are known in the prior art and are designed in the manner of a Köhler illumination, an auxiliary lens arrangement is arranged downstream of the collector lens arrangement 22. Starting from such prior art, the pancratic system 26 used according to the invention is part of the auxiliary lens arrangement or forms the auxiliary lens arrangement.

The lighting arrangement according to the invention is particularly suitable for diagnostic or surgical microscopes. However, it can also be used in any other microscopes.

While this invention has been described as having a preferred design, it is understood that it is capable of further modifications, and uses and/or adaptations of the invention and following in general the principle of the invention and including such departures from the present disclosure as come within the known or customary practice in the art to which the invention pertains, and as may be applied to the central features hereinbefore set forth, and fall within the scope of the invention.

What is claimed is:

1. A lighting arrangement for a diagnostic or surgical microscope, the lighting arrangement comprising:
a) a light source, wherein the lighting arrangement defines an illuminating beam path;
b) an illumination optics, the illumination optics is or can be arranged between the light source, and an objective of the microscope, which defines an imaging beam path;
c) in the beam path of the light source at least one deflection element is provided for coupling in of the illuminating beam path into the imaging beam path;
d) the illumination optics has at least one pancratic system;
e) the lighting arrangement has, in the manner of a Köhler illumination, a collect lens arrangement which is arranged downstream of the light source in the beam direction of the light source, a field diaphragm arranged downstream of the collector lens arrangement, and an auxiliary lens arrangement arranged downstream of the field diaphragm; and
f) the auxiliary lens arrangement has at least one pancratic system.

2. The lighting arrangement according to claim 1, wherein:
a) the light source has at least one light-emitting diode.

3. The lighting arrangement according to claim 1, wherein:
a) the lighting arrangement has an optical device for reducing reflections of the illumination light on the objective of the microscope.

4. The lighting arrangement according to claim 3, wherein:
a) the optical device for reducing reflections is or can be arranged in the beam direction of the light source between the illumination optics and the objective of the microscope.

5. The lighting arrangement according claim 3, wherein:
a) the optical device for reducing reflections has at least one pinhole diaphragm.

6. The lighting arrangement according to claim 5, wherein:
a) the pinhole diaphragm has at least one opening for the illumination main beam path of the lighting arrangement.

7. The lighting arrangement according to claim. 1, wherein:
a) the deflection element is a prism or a mirror.

8. The lighting arrangement according to claim 1, wherein:
a) the field diaphragm is formed as an iris diaphragm.

9. A microscope including the lighting arrangement according to claim 1.

10. A lighting arrangement for a diagnostic or surgical microscope, the lighting arrangement comprising:
a) a light source, wherein the lighting arrangement defines an illuminating beam path;
b) an illumination optics, the illumination optics is or can be arranged between the light source, and an objective of the microscope, which defines an imaging beam path;
c) in the beam path of the light source at least one prism or mirror is provided for coupling in of the illuminating beam path into the imaging beam path;
d) the illumination optics has at least one pancratic system;
e) the lighting arrangement has, in the manner of a Köhler illumination, a collector lens arrangement which is arranged downstream of the light source in the beam direction of the light source, a field diaphragm arranged downstream of the collector lens arrangement, and an auxiliary lens arrangement arranged downstream of the field diaphragm; and
f) the auxiliary lens arrangement has at least one pancratic system.

11. The lighting arrangement according to claim 10, wherein:
a) the light source has at least one light-emitting diode.

12. The lighting arrangement according to claim 10, wherein:
a) the lighting arrangement has an optical device for reducing reflections of the illumination light on the objective of the microscope.
